(19) United States
(12) Patent Application Publication
(10) Pub. No.: US 2006/0264505 A1
(43) Pub. Date: Nov. 23, 2006

(54) Dermatological compositions

(75) Inventors: Karl F. Popp, Schodack Landing, NY (US); Ronald J. Santelli, Newark, NY (US)

Correspondence Address: Gary M. Nath;NATH & ASSOCIATES PLLC, 112 S. West Street, Alexandria, VA, 22314, US

(73) Assignee: STIEFEL LABORATORIES, INC., Coral Gables, FL (US)

(21) Appl. No.: 11/493,589

(22) Filed: Jul. 27, 2006

Related U.S. Application Data
(63) Continuation-in-part of application No. 10/702,782, filed on Nov. 7, 2003.

(51) Int. Cl. A61K 31/22; A61K 31/17

(52) U.S. Cl. 514/547; 514/588

(57) ABSTRACT

Topical dermatological compositions comprising urea as an active ingredient, a vegetable oil or a derivative thereof, water, and conventional excipients. These compositions are used for topical medical applications, particularly to treat various skin disorders.

This application is a continuation-in-part of U.S. application Ser. No. 10/702,782, filed Nov. 7, 2003, the contents of which are hereby incorporated by reference in their entirety.

### FIELD OF THE INVENTION

The present inventive subject matter relates to topical dermatological compositions comprising urea as an active ingredient, a vegetable oil or vegetable oil derivative, water, and conventional excipients. These compositions are used for topical medical applications, particularly to treat various skin disorders.

### BACKGROUND OF THE INVENTION

Urea has long been recognized as a cosmetic ingredient in formulations acting as a humectant and moisturizer. There have been reports of keratolytic activity attributed to urea with the ability at high concentrations to solubilize and denature protein. High concentrations of urea are also known to have a mild, antibacterial effect.

Many topical compositions containing urea as an active ingredient are known in the art. These compositions tend to comprise an oil-in-water emulsion, or an oil base. The oil used in these prior art compositions is traditionally a mineral oil, or some other oil of an oleaginous nature derived from petroleum.

For example, U.S. Pat. No. 5,919,470 describes a composition containing urea combined with skin protectants of an oleaginous nature derived from petroleum and suitable emulsifiers and thickeners. In preferred embodiments, the disclosed compositions contain a combination of semi-solid and liquid petroleum fractions.

However, these petroleum based oils such as mineral oil lack functional-based OH groups, i.e. have no “handle”. Accordingly, mineral oil is not readily absorbed by the skin and tends to block skin pores. Further, mineral oil tends to be comedogenic in that it causes or worsens a build-up of dead cells in the follicles that leads to the development of blackheads.

Additionally, petroleum based oils are greasy in nature and are difficult to wash off the skin. Accordingly, petroleum based oils can prevent or occlude moisture evaporation from the skin.

For these reasons, topical compositions containing a vegetable oil or vegetable oil derivative in various forms (i.e., fluid, semisolid, or solid), rather than a mineral oil, base or carrier for a urea active ingredient have long been sought in the art. However, commercially feasible urea compositions comprising a vegetable oil base have previously been unattainable due to stability problems inherent in using vegetable oil or vegetable oil derivatives as a topical base. In particular, previous compositions with a vegetable oil base required a preservative to inhibit the growth of bacteria in the composition. The use of a preservative, however, conveyed the potential side effects of increased irritation and decreased patient compliance to these compositions.

Accordingly, there remains a need in the art for a stable topical urea composition containing a vegetable-derived oil rather than a mineral oil base in order to maximize the skin treating and moisturizing effects of the active urea ingredient. There further remains a need for urea compositions that would increase patient compliance due to a decreased greasy skin feel. The present inventive subject matter addresses these needs.

### SUMMARY OF THE INVENTION

The present inventive subject matter relates to a dermatological composition suitable for topical administration comprising:

about 5 to about 60% by weight of an active ingredient consisting of urea;

about 3 to about 45% by weight of a vegetable oil or a derivative thereof;

water; and

remaining amounts of one or more dermatologically acceptable agents.

In a preferred embodiment, the present inventive subject matter relates to a dermatological composition suitable for topical administration comprising an oil-in-water emulsion comprising:

an oil phase comprising about 3 to about 20% by weight of the overall weight of the composition of a vegetable oil or a derivative thereof and at least one emulsifier; and

an aqueous phase comprising about 30 to about 60% by weight of the overall weight of the composition of an active ingredient consisting of urea and about 15 to about 55% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In another preferred embodiment, the present inventive subject matter relates to a dermatological composition suitable for topical administration comprising an oil-in-water emulsion comprising:

an oil phase comprising about 15 to about 45% by weight of the overall weight of the composition of a vegetable oil or a derivative thereof and at least one emulsifier; and

an aqueous phase comprising about 40 to about 55% by weight of the overall weight of the composition of an active ingredient consisting of urea and about 25 to about 40% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In yet another preferred embodiment, the present inventive subject matter relates to a dermatological composition suitable for topical administration comprising an oil-in-water emulsion comprising:

an oil phase comprising about 3 to about 20% by weight of the overall weight of the composition of a vegetable oil or a derivative thereof and at least one emulsifier; and

an aqueous phase comprising about 21 to about 50% by weight of the overall weight of the composition of an active ingredient consisting of urea and about 35 to about 55% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In still another preferred embodiment, the present inventive subject matter relates to a method of treating a skin disorder in a mammal, comprising topically administering to skin of a mammal in need thereof a therapeutically effective amount of a dermatological composition suitable for topical administration comprising an oil-in-water emulsion comprising:

an oil phase comprising about 3 to about 20% by weight of the overall weight of the composition of a vegetable oil or a derivative thereof and at least one emulsifier; and

an aqueous phase comprising about 21 to about 60% by weight of the overall weight of the composition of an active ingredient consisting of urea and about 35 to about 55% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In a further preferred embodiment, the present inventive subject matter relates to a method of treating or preventing dry skin in a mammal, comprising topically administering to skin of a mammal in need thereof a therapeutically effective amount of a dermatological composition suitable for topical administration comprising an oil-in-water emulsion comprising:

an oil phase comprising about 3 to about 20% by weight of the overall weight of the composition of a vegetable oil or a derivative thereof and at least one emulsifier; and

an aqueous phase comprising about 21 to about 60% by weight of the overall weight of the composition of an active ingredient consisting of urea and about 35 to about 55% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In yet another preferred embodiment, the present inventive subject matter relates to a dermatological composition suitable for topical administration comprising an oil-in-water emulsion comprising:

an oil phase comprising about 15 to about 45% by weight of the overall weight of the composition of a vegetable oil or a derivative thereof and at least one emulsifier; and

an aqueous phase comprising about 5 to about 20% by weight of the overall weight of the composition of an active ingredient consisting of urea and about 45 to about 90% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In still another preferred embodiment, the present inventive subject matter relates to a method of treating a skin disorder in a mammal, comprising topically administering to skin of a mammal in need thereof a therapeutically effective amount of a dermatological composition suitable for topical administration comprising an oil-in-water emulsion comprising:

an oil phase comprising about 15 to about 45% by weight of the overall weight of the composition of a vegetable oil or a derivative thereof and at least one emulsifier; and

an aqueous phase comprising about 5 to about 20% by weight of the overall weight of the composition of an active ingredient consisting of urea and about 45 to about 90% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In a further preferred embodiment, the present inventive subject matter relates to a method of treating or preventing dry skin in a mammal, comprising topically administering to skin of a mammal in need thereof a therapeutically effective amount of a dermatological composition suitable for topical administration comprising an oil-in-water emulsion comprising:

an oil phase comprising about 15 to about 45% by weight of the overall weight of the composition of a vegetable oil or a derivative thereof and at least one emulsifier; and

an aqueous phase comprising about 5 to about 20% by weight of the overall weight of the composition of an active ingredient consisting of urea and about 45 to about 90% by weight of the overall weight of the composition of water,

wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

In another preferred embodiment, the present inventive subject matter relates to a dermatological composition suitable for topical administration comprising:

about 5 to about 50% by weight of an active ingredient consisting of urea;

about 3 to about 45% by weight of a vegetable oil or a derivative thereof;

water; and

remaining amounts of one or more dermatologically acceptable agents.

In yet another preferred embodiment, the present inventive subject matter relates to a method of treating a skin disorder in a mammal, comprising topically administering to skin of a mammal in need thereof a therapeutically effective amount of a dermatological composition suitable for topical administration comprising:

about 5 to about 50% by weight of an active ingredient consisting of urea;

about 3 to about 45% by weight of a vegetable oil or a derivative thereof;

water; and

remaining amounts of one or more dermatologically acceptable agents.

In another preferred embodiment, the present inventive subject matter relates to a method of treating or preventing dry skin in a mammal, comprising topically administering to skin of a mammal in need thereof a therapeutically effective amount of a dermatological composition suitable for topical administration comprising:

about 5 to about 50% by weight of an active ingredient consisting of urea;

about 3 to about 45% by weight of a vegetable oil or a derivative thereof;

water; and

remaining amounts of one or more dermatologically acceptable agents.

In yet another further preferred embodiment, the present inventive subject matter relates to a process for preparing a dermatological composition suitable for topical administration comprising an emulsion, said process comprising:
1) preparing an aqueous phase comprising about 5 to about 60% by weight of the overall weight of the composition of an active ingredient consisting of urea, and about 5 to about 90% by weight of the overall weight of the composition of water,
2) heating said aqueous phase to a temperature of about 60 to about 80° C. while mixing;
3) preparing an oil phase comprising about 3 to about 45% by weight of the overall weight of the composition of a vegetable oil or a derivative thereof and at least one emulsifier;
4) combining said oil phase with said aqueous phase while mixing at a temperature of about 60 to about 80° C. to obtain a homogenous emulsion;
5) cooling said emulsion to a temperature of about 15 to about 30° C.; and
6) recovering a topical emulsion dermatological composition.

### DETAILED DESCRIPTION OF THE INVENTION

### DEFINITIONS

The term “administering” as used herein refers to any method which, in sound medical practice, delivers the composition to a subject in such a manner so as to be effective in the treatment of a dermatological disorder. The compositions are preferably administered such that they cover the entire area to be treated.

The phrase “effective amount”, as used herein, means an amount of a composition or component thereof sufficient enough to positively modify the disorder to be treated but low enough to avoid serious side effects, within the scope of sound medical advice. Effective amounts will vary with the particular disorder or disorders being treated, the severity of the disorder, the duration of the treatment, the specific components of the composition being used, and like factors as are known by health-care providers, including physicians.

As used herein, “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also by used, as can guanidine and its derivatives, in particular its alkylation products. However, it is also possible to employ as salt-forming agents, for example, low molecular weight alkylamines such as methylamine, ethylamine, or triethylamine. Suitable salts for the compounds to be employed according to the present inventive subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the.basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, “treating” or “treatment” means the prevention or reduction of severity of symptoms or effect of a dermatological disorder, disease, infection, allergy, reaction, or other dermatological condition.

Other terms as used herein are meant to be defined by their well-known meanings in the art.

### Topical Dermatological Compositions

The present inventive subject matter pertains to dermatological composition suitable for topical administration comprising:

about 5 to about 60% by weight of an active ingredient consisting of urea;

about 3 to about 45% by weight of a vegetable oil or a derivative thereof;

water; and

remaining amounts of one or more dermatologically acceptable agents.

In a preferred embodiment, the present inventive dermatological compositions comprise about 5 to 90% by weight water. In a particularly preferred embodiment, the present inventive dermatological compositions comprise about 15 to about 55% by weight water. In an alternative particularly preferred embodiment, the present inventive dermatological compositions comprise about 25 to about 40% by weight water.

The present inventive topical dermatological compositions are unique in that they employ a vegetable oil or a derivative thereof as a vehicle for the urea active ingredient. Accordingly, these compositions have a decided advantage over previous compositions containing a mineral oil vehicle in that they provide an increased absorption of the urea into the skin to which the composition is applied.

The present compositions, then, provide an enhanced effect in treating skin disorders treatable with urea over these previous compositions. Accordingly, the present urea-containing dermatological compositions do not require an additional ingredient as an essential component to enhance the effect of the urea on skin disorders. As fewer ingredients are present in these compositions, the chances of a patient having an adverse reaction to the medicine will decrease. The present inventive urea-containing dermatological compositions, then, are expected to irritate the skin of fewer patients than do the previously known urea compositions containing additional ingredients required due to the presence of a mineral oil.

Further, because the present inventive compositions contain a vegetable oil or vegetable oil derivative rather than a mineral oil, these compositions are readily absorbed by the skin and do not block skin pores, allowing the skin to breathe. This enhanced “breathing” can be evidenced by the enhanced evaporation of moisture from the skin, resulting in a pleasing cooling effect. This improved skin absorption is also partly due to a decreased comedogenicity, or build-up of dead cells in the follicles, resulting from the use of vegetable oils or vegetable oil derivatives over mineral oils. This further enhances the effectiveness of the urea active ingredient, as well as increases the ease of use to the patient.

Additionally, the vegetable oils or vegetable oil derivatives included in the present inventive compositions are very similar to human sebum, which is beneficial for skin that does not produce sufficient amounts of sebum. This provides further therapeutic benefits over previously known compositions containing mineral oil.

The present inventive compositions also provide an enhanced skin-feel to a patient being treated. Dermatological compositions containing petroleum-based oils are greasy in nature. These greasy products are less acceptable to patients as they may stain clothing and are difficult to wash off the skin. Accordingly, it would be expected that the present inventive compositions would result in an increased patient compliance with the strict daily regimen of topical urea administration since the use of a vegetable oil or vegetable oil derivative base reduces this greasy feel.

The present inventive compositions are remarkably stable considering the presence of vegetable oil or a vegetable oil derivative as the oil base. Accordingly, these compositions solve long felt difficulties in formulating urea compositions with a vegetable oil. For example, the present inventive compositions do not require a preservative as an essential component to inhibit the growth of bacteria within the compositions. Accordingly, the present inventive compositions avoid the disadvantageous side effects, such as increased irritation, previously observed with certain vegetable oil based compositions containing a preservative as an essential component.

The selection of the specific excipients and amounts thereof used in the present inventive compositions, as well as the preparation of compositions having a specific designated pH in the form of a designated emulsion, conveys these unique stability characteristics to the present inventive dermatological compositions.

The present inventive compositions are preferably formed as an emulsion having an oil phase and an aqueous phase. Non-limiting examples of specific types of emulsions contemplated herein include an oil-in-water emulsion, a water-in-oil emulsion, an oil-in-water-in-oil emulsion, and a water-in-oil-in-water emulsion. The formation of a specific type of emulsion will depend on the specific surfactant system and amount of vegetable oil or vegetable oil derivative used in forming the composition.

In a preferred embodiment, the compositions are formed as an oil-in-water emulsion. Preferably, the oil phase of this emulsion comprises a vegetable oil or derivative thereof and an emulsifier to aid in formation of the emulsion. It is noted, however, that during formation of this specific oil-in-water emulsion, other emulsion systems may briefly exist in a localized area before being incorporated into the remainder of the composition as an oil-in-water emulsion.

Since the preferred emulsion is an oil-in-water emulsion having water as the major component, the final composition will have a pH mirroring that of the aqueous phase. The pH of the aqueous phase, and of the final composition, is adjusted to range from about 3 to about 11. In a preferred embodiment, the pH of the final composition is adjusted to range from about 5 to about 10.5. In a particularly preferred embodiment, the pH of the final composition is adjusted to range from about 7 to about 10. This pH is specifically designed to maximize the stability of the vegetable oil or derivative thereof forming the oil phase. It is noted in this regard that a higher pH, i.e., a pH of about 7 or greater, is desired as a greater amount of urea is present in the composition, e.g. about 40% by weight urea.

Active Ingredient

The present inventive dermatological compositions contain about 5 to about 60% by weight of urea as the sole active ingredient. In a preferred embodiment, the present inventive compositions contain about 30 to about 60% by weight of the urea active ingredient.

In a particularly preferred embodiment, the present inventive compositions contain about 45 to about 55% by weight of the urea active ingredient. Compositions containing about 40 to about 55% by weight of the urea active ingredients are further preferred, while compositions containing about 47 to about 53% by weight of the urea active ingredient are especially preferred in this regard.

In this regard, it is noted that formulations containing about 5% by weight urea are typically used as cosmetics. As the amount of urea in the formulation rises to the level of about 10%, the formulation exhibits certain limited dermatological treatment aspects. At percentages of about 10% or greater, the urea serves as an active ingredient in the formulation, rendering the formulation available for use as a dermatological treatment product.

This urea active ingredient is intended to treat various skin disorders generally due to its abilities as a humectant, a moisturizer, and a keratolytic substance, as well as due to its antibacterial effects.

The urea active ingredient is preferably present in the aqueous phase of the instant emulsion compositions. This maximizes both the overall stability of the composition and the dermatological effectiveness of the urea active ingredient. In an alternative embodiment, the urea can be present in the oil phase of the instant emulsion compositions.

Vegetable Oils

The oil phase of the present inventive dermatological compositions is made up of about 3 to about 45% by weight, of the overall weight of the composition, of a vegetable oil or a derivative thereof. In a preferred embodiment, the present inventive compositions contain about 3 to about 20% by weight of the overall weight of the composition of a vegetable oil or a derivative thereof.

In a particularly preferred embodiment, the present inventive compositions contain about 5 to about 15% by weight of the vegetable oil or a derivative thereof. Compositions containing about 5 to about 10% by weight of the vegetable oil or a derivative thereof are especially preferred in this regard.

In an alternative particularly preferred embodiment, the present inventive compositions contain about 15 to about 45% by weight of the vegetable oil or a derivative thereof. Compositions containing about 20 to about 35% by weight of the vegetable oil or a derivative thereof are especially preferred in this regard.

The use of a vegetable oil rather than a mineral oil permits the present inventive compositions to be readily absorbed by the skin and to not block skin pores, allowing the skin to breathe and resulting in the enhanced evaporation of moisture from the skin. Additionally, the vegetable oils or vegetable oil derivatives included in the present inventive compositions are very similar to human sebum, which is beneficial for skin that does not produce sufficient amounts of sebum. Further, the use of vegetable oils or derivatives thereof results in dermatological compositions that are not greasy.

The vegetable oil or derivative thereof useful in the present compositions can take the physical form of a fluid, semi-solid, solid, or any other form in which vegetable oils are presently known as available. Preferably, the vegetable oil or derivative thereof is selected from the group consisting of castor oil, sunflower oil, a liquid fraction of karite butter, cottonseed oil, canola oil, corn oil, hydrogenated vegetable oil, peanut oil, sesame oil, soybean oil, palm oil, coconut oil, rapeseed oil, safflower oil, cocoa butter, linseed oil, olive oil, almond oil, avocado oil, citrus seed oil, cohune oil, tallow, oat oil, palm kernel oil, rice bran oil, tucum oil, babassu oil, derivatives thereof, and mixtures thereof. In a particularly preferred embodiment, the vegetable oil is castor oil or a derivative thereof. Other vegetable oils or vegetable oil derivatives known in the art as useful in topical dermatological compositions are additionally contemplated as within the scope of the present inventive subject matter.

Dermatologically Acceptable Agents

The present inventive dermatological compositions additionally contain remaining amounts of one or more dermatologically acceptable agents. Preferred, non-limiting examples of dermatologically acceptable agents useful in the present inventive compositions are those selected from the group consisting of emulsifiers, surfactants, suspending agents, antioxidants, chelates, preservatives, emollients, humectants, fluid alkyl alcohols, thickening agents, polysiloxanes, modified polysiloxanes, pH modifiers, and mixtures thereof.

In this regard, the additional fluid alkyl alcohol may be used as a solvent for the present compositions in place of water. Likewise, the modified polysiloxanes can be used as a carrier for a gel form of the present compositions.

Emulsifiers

The emulsion of the present inventive dermatological compositions is preferably formed using at least one emulsifier. Non-limiting examples of suitable emulsifiers useful in the present inventive dermatological compositions include straight or branched chain fatty acids, polyoxyethylene sorbitan fatty acid esters, sorbitan fatty acid esters, propylene glycol stearate, glyceryl stearate, polyethylene glycol, polyethylene glycol stearates, fatty alcohols, polymeric ethylene oxide-propylene oxide block polymers, derivatives thereof, pharmaceutically acceptable salts thereof, and mixtures thereof.

In a particularly preferred embodiment, the emulsifier is selected from the group consisting of cetyl alcohol, laureth-4, glyceryl stearate and polyethylene glycol-100/glyceryl stearate, polyethylene glycol 40 stearate, and mixtures thereof. In yet another preferred embodiment, the emulsifier is present in a phase of said emulsion selected from the group consisting of said oil. phase, said aqueous phase, and a combination thereof.

Surfactants

Non-limiting examples of surfactants useful in the present inventive compositions include nonionic surfactants, anionic surfactants, amphoteric surfactants, cationic surfactants, and mixtures thereof.

Preferred, non-limiting examples of amphoteric surfactants useful in the present inventive dermatological compositions are those selected from the group consisting of alkyl betaines, alkylamidobetaines, aminopropionates, iminodipropionates, aminoglycinates, imidazolinium betaines, sulfobetaines, and mixtures thereof.

Specific, non-limiting examples of preferred amphoteric surfactants useful in the present inventive dermatological compositions are those selected from the group consisting of sodium 3-dodecyl-aminopropionate, sodium 3-dodecylaminopropane sulfonate, sodium lauroamphoacetate, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and mixtures thereof.

Similarly, preferred, non-limiting examples of anionic surfactants useful in the present inventive dermatological compositions are those selected from the group consisting of alkyl sulfates, alkyl ethoxylated sulfates, beta-alkyloxy alkane sulfonates, alkyl ether sulfates, alkyl glyceryl ether sulfonates, alkyl ether carboxylates, acyl isethionates, acyl sarcosinates, acyl taurines, succinates, alkali metal, ammonium, or alkanolammonium salts thereof, and mixtures thereof.

Specific, non-limiting examples of preferred anionic surfactants useful in the present inventive dermatological compositions are those selected from the group consisting of ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarboxyethyl)-N-octadecylsulfosuccinnate, diamyl ester of sodium sulfosuccinic acid, dihexyl ester of sodium sulfosuccinic acid, dioctyl esters of sodium sulfosuccinic acid, docusate sodium, and mixtures thereof. A particularly preferred anionic surfactant useful in the present inventive compositions is polyoxyl stearate.

Specific, non-limiting examples of preferred cationic surfactants useful in the present inventive dermatological compositions include those selected from the group consisting of behenyl trimethyl ammonium chloride, bis(acyloxyethyl) hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowdimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquaternium, stearalkonium chloride, sterayl dimethylbenzyl ammonium chloride, stearyl trimethyl ammonium chloride, trimethylglycine, and mixtures thereof.

Specific, non-limiting examples of preferred nonionic surfactants useful in the present dermatological compositions include those selected from the group consisting of polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and mixtures thereof. A particularly preferred nonionic surfactant useful in the present inventive compositions is laureth-4.

In a particularly preferred embodiment, the present inventive dermatological compositions contain at least two surfactants. In a more particularly preferred embodiment, these surfactants are selected from the group consisting of polyoxyl stearate, laureth-4, and mixtures thereof. In yet another preferred embodiment, the surfactant(s) are present in the oil phase, the aqueous phase, or a combination thereof of the present inventive dermatological compositions.

Suspending Agents

The present inventive dermatological compositions may further contain a suspending agent. Non-limiting examples of suitable suspending agents useful in the present inventive dermatological compositions include alginic acid, bentonite, carbomer, carboxymethylcellulose and salts thereof, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and mixtures thereof.

In a particularly preferred embodiment, the suspending agent is magnesium aluminum silicate. The present inventive compositions may comprise about 0.5 to about 5% by weight of such a preferred suspending agent. In another particularly preferred embodiment, the present inventive compositions may comprise about 1 to about 3.5% by weight of such a preferred suspending agent.

In yet another preferred embodiment, the suspending agent is present in the oil phase, the aqueous phase, or a combination thereof of the present inventive dermatological compositions.

Antioxidants

The present inventive dermatological compositions may further contain an antioxidant. Preferred non-limiting examples of antioxidants useful in the present inventive compositions include those selected from the group consisting of butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and mixtures thereof.

Chelating Agents

The present inventive dermatological compositions may further contain a chelating agent. Preferred non-limiting examples of chelating agents useful in the present inventive compositions include those selected from the group consisting of EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosponic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11] pentatriacontane hexahydrobromide, triethylenetetramine-N,N,N′,N″,N′″,N′″-hexaacetic acid, pharmaceutically acceptable salts thereof, and mixtures thereof.

Emollients

The present inventive dermatological compositions may further contain an emollient. While these emollients may be present in certain embodiments of the present compositions, that are not critical components of these compositions. Further, when present, these emollients only comprise a small percentage of the present compositions; the compositions do not need to carry high levels of the emollient.

Preferred non-limiting examples of emollients useful in the present inventive compositions include those selected from the group consisting of myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and mixtures thereof.

Humectants

The present inventive dermatological compositions may further contain a humectant. Preferred non-limiting examples of humectants useful in the present inventive compositions include glycerin, butylene glycol, propylene glycol, sorbitol, and triacetin.

pH Modifiers

The present inventive dermatological compositions may further contain sufficient amounts of at least one pH modifier to ensure that the composition has a final pH of about 3 to about 11. The preparation of an overall composition having this specific pH in the form of a designated emulsion conveys the unique stability characteristics to the present inventive dermatological compositions.

Preferred non-limiting examples of pH modifiers useful to impart the desired pH to the present inventive compositions are those selected from the group consisting of sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and a mixture thereof. The pH modifiers sodium hydroxide and citric acid are most preferred in this regard.

Urea-Modifying Agents

The present inventive dermatological compositions may further comprise a urea-modifying agent. This urea-modifying agent acts to enhance the effects and curative action of the primary urea active ingredient on the skin. Further, the urea-modifying agent moisturizes the skin, reducing normal side effects of urea use such as stinging, burning, itching, and irritation of the skin. Accordingly, the addition of the urea-modifying agent to the present compositions may enhance the beneficial effects of the urea compositions and improve patient compliance with a prescribed treatment regimen.

Preferred, non-limiting examples of these urea-modifying agents are selected from the group consisting of a pyrrolidone carboxylate salt, dimethyl isosorbide, polyethylene glycol, propylene glycol, hexylene glycol, butylenes glycol, triethaline glycol, dipropylene glycol, glycerin, derivatives thereof, and mixtures thereof. A particularly preferred, non-limiting example of a pyrrolidone carboxylate salt in this regard is sodium pyrrolidone carboxylate.

In a particularly preferred embodiment, the present inventive dermatological compositions are formulated in a lotion, cream, ointment, shampoo, gel, aerosol, solution (solubilized system), paste, suspension, skin cleanser, bar (such as a soap bar), or other pharmaceutically acceptable topical dosage form.

Methods of Treatment

The present inventive subject matter additionally pertains to a method of treating a skin disorder in a mammal, comprising topically administering to skin of a mammal in need thereof a therapeutically effective amount of a dermatological composition suitable for topical administration comprising:

about 5 to about 60% by weight of an active ingredient consisting of urea;

about 3 to about 45% by weight of a vegetable oil or a derivative thereof;

water; and

remaining amounts of one or more dermatologically acceptable agents.

Several specific skin disorders may be treated according to the present inventive methods. Exemplary among these skin disorders are cracked skin, disturbed barrier function, dry skin, eczema, icthyotic atopic eczema, acthyotic skin, subacute and chronic atopic eczema, xerosis, rough skin, dermatitis, psoriasis, ichthyosis, keratosis, keratoderma, corns, calluses, scales, nummular dermatitis, lichen simplex chronicus, pityriasis rosea, dandruff, acne, pruritus, age spots, lentigines, melasmas, wrinkles, warts, blemished skin, hyperpigmented skin, hyperkeratotic skin, inflammatory dermatoses, skin changes associated with aging, skin requiring cleansers, and combinations thereof.

Also included among the skin disorders that can be treated by the present inventive compositions are disorders due to changes in normal keratinization, epidermal formation or pilosebaceous function, such as acne, psoriasis, seborrhea, ingrown hairs and pseudofolliculitis barbae, and cutaneous infections. Other skin disorders known to those of ordinary skill in the art as effectively treatable by a topical composition are further contemplated as within the scope of the present inventive subject matter.

In a preferred embodiment, the skin disorder to be treated or prevented according to the present inventive methods is dry skin. The administration of the present inventive compositions to the skin moisturizes the skin. Accordingly, the present inventive methods improve the moisture content of skin to which the present topical compositions are administered.

In another preferred embodiment, the present inventive compositions can be used to treat dermatological disorders resulting in visible lesions. Examples of such disorders include acne, cutaneous infections, psoriasis and other disorders of the cutaneous and pilosebaceous unit or the process of keratogenesis. Visible lesions include closed comedones, open comedones, red or pustular-looking inflamed papules, pustules, nodules and cysts of acne or cutaneous infection; visible ingrown hairs of pseudofolliculitis barbae; visible scales of seborrhea, ichthyosis and psoriasis; and the like. Visible lesions can be due to obstruction of follicular ducts, thickened sebum, bacterial infection, or a combination thereof. Accordingly, the present inventive compositions can be used to prevent obstruction of follicular ducts, to reopen a duct if it has become blocked, to combat thickened sebum, to combat bacterial infection, or a combination thereof. Treatment of visible lesions can be evaluated based on the effectiveness of the treatment in reducing the number and severity of visible lesions. Any reduction in number or severity of visible lesions as a result of administration of the present inventive composition would be considered treatment of visible lesions.

In yet another preferred embodiment, the present inventive compositions can be used to treat pre-emergent lesions. As used herein, “pre-emergent lesions” refer to non-visible lesions present within the skin prior to eruption of visible lesions on the surface of the skin. Like visible lesions, pre-emergent lesions can be due to obstruction of follicular ducts, thickened sebum, bacterial infection, or a combination thereof. Accordingly, the present inventive compositions can be used to treat pre-emergent lesions by preventing obstruction of follicular ducts, reopening a duct if it has become blocked, combating thickened sebum, combating bacterial infection, or a combination thereof. While pre-emergent lesions are insufficiently visible to be graded in conventional clinical studies, their presence within the skin can be discerned by the tactile sense of feel and/or by pain and tension within the skin. Any reduction in number of locations within the skin in which pre-emergent lesions exist as a result of administration of the present inventive compositions would be considered treatment of pre-emergent lesions. Similarly, any reduction in the severity of the symptoms of a pre-emergent lesion as a result of administration of the present inventive compositions would be considered treatment of the pre-emergent lesion.

In still another embodiment, the present inventive compositions can be used to treat acne. As used herein, “acne” means a disorder of the skin caused by inflammation of skin glands or hair follicles. The compositions of the invention can be used to treat acne at early pre-emergent stages or later stages where lesions from acne are visible.

Early pre-emergent stages of acne usually begin with an excessive secretion of sebum or dermal oil from the sebaceous glands located in the pilosebaceous apparatus. Sebum reaches the skin surface through the duct of the hair follicle. The presence of excessive amounts of sebum in the duct and on the skin tends to obstruct or stagnate the normal flow of sebum from the follicular duct, thus producing a thickening and solidification of the sebum to create a solid plug known as a comedone.

In the normal sequence of developing acne, hyperkeratinazation of the follicular opening is stimulated, thus completing blocking of the duct. The usual results are papules, pustules, or cysts, often contaminated with bacteria, which cause secondary infections.

Acne is characterized particularly by the presence of comedones, inflammatory papules, or cysts. The appearance of acne may range from slight skin irritation to pitting and even the development of disfiguring scars. Accordingly, the present inventive compositions can be used, but not limited, to treat skin irritation, pitting, development of scars, comedones, inflammatory papules, cysts, hyperkeratinazation, and thickening and hardening of sebum associated with acne.

Methods of Production

The present inventive subject matter further relates to a process for preparing a dermatological composition suitable for topical administration comprising an emulsion, said process comprising:

1) preparing an aqueous phase comprising about 5 to about 60% by weight of the overall weight of the composition of an active ingredient consisting of urea, and about 5 to about 90% by weight of the overall weight of the composition of water,
2) heating said aqueous phase to a temperature of about 60 to about 80° C. while mixing;
3) preparing an oil phase comprising about 3 to about 45% by weight of the overall weight of the composition of a vegetable oil or a derivative thereof and at least one emulsifier;
4) combining said oil phase with said aqueous phase while mixing at a temperature of about 60 to about 80° C. to obtain a homogenous emulsion;
5) cooling said emulsion to a temperature of about 15 to about 30° C.; and
6) recovering a topical emulsion dermatological composition.

In a preferred embodiment of the present inventive subject matter, the aqueous phase is prepared by first mixing the active ingredient in purified water at a temperature of about 20 to about 30° C. before adding at least one suspending agent to the mixture. In a particularly preferred embodiment, the active ingredient and water are mixed for at least 20 minutes before the at least one suspending agent is added. Once the at least one suspending agent has been added to the aqueous phase, the mixture is preferably mixed until the aqueous phase is uniform in appearance.

Accordingly, the aqueous phase, in total, can be prepared by mixing the active ingredient, water, and at least one suspending agent at a temperature of about 20 to about 30° C. After the aqueous phase has been suitably mixed, it may be heated to a temperature of about 65 to about 75° C.

In another preferred embodiment of the present inventive subject matter, the oil phase is prepared by heating a vegetable oil or derivative thereof, at least one emulsifier, and at least two surfactants at a temperature of about 60 to about 80° C. until melted. Once each of these ingredients have melted, the oil phase is mixed until it is uniform while maintained at a temperature of about 60 to about 80° C.

Accordingly, the process can additionally comprise adding an additional component selected from the group consisting of at least one suspending agent, at least two surfactants, and combinations thereof to either or both of said aqueous phase and said oil phase in either or both of steps 1) and 3).

Once each of the aqueous phase and the oil phase have been separately prepared, the oil phase is combined with the aqueous phase and maintained at a temperature of about 60 to about 80° C. while mixing for at least 20 minutes, or until uniform in appearance.

This process preferably forms compositions comprising an emulsion having an oil phase and an aqueous phase. Non-limiting examples of specific types of emulsions that can be made according to this process include an oil-in-water emulsion, a water-in-oil emulsion, an oil-in-water-in-oil emulsion, and a water-in-oil-in-water emulsion. The formation of a specific type of emulsion will depend on the specific surfactant system and amount of vegetable oil or vegetable oil derivative used in the process.

In a preferred embodiment, the process will form compositions that are oil-in-water emulsions. It is noted, however, that during the process of forming this specific oil-in-water emulsion, the process may briefly form other emulsion systems in a localized area before they are incorporated into the remainder of the composition as an oil-in-water emulsion. For example, when the oil phase is first added to the aqueous phase, mini water-in-oil emulsions will very briefly form in localized spots of the overall composition. These mini-emulsions will quickly dissipate and become incorporated as a part of the overall oil-in-water emulsion.

This particular preparation process is a non-limiting example of a possible process that can be used to prepare the present inventive compositions. Other processes capable of preparing these compositions are further contemplated herein. Further, the individual phases of the present compositions (for example aqueous and oil phases) can be prepared sequentially in any order or concurrently; it is not a necessary aspect of the present inventive processes that the aqueous phase be prepared before the oil phase is prepared. Additionally, the present compositions can be prepared according to either a batch process or continuously.

Further contemplated as within the scope of the present inventive subject matter are pharmaceutical compositions produced according to the above-described process. If produced according to the present inventive process, these compositions exhibit chemical and physical stability suitable for topical administration.

The compositions produced according to these processes can further be used in a lotion, cream, ointment, shampoo, gel, aerosol, solution (solubilized system), paste, suspension, skin cleanser, bar (such as a soap bar), or other pharmaceutically acceptable topical dosage form. These compositions can be placed in a suitable containment vessel comprising a product contact surface composed of a material selected from the group consisting of glass, plastic, steel, stainless steel, aluminum, Teflon, polymeric structure, ceramic structure, alloys, and mixtures thereof. These containment vessels are used to facilitate manufacturing, handling, processing, packaging, storage, and administration of said composition.

### Dosage

Appropriate dosage levels for the urea active agent contemplated in the present inventive subject matter are well known to those of ordinary skill in the art. Dosage levels on the order of about 0.001 mg to about 5,000 mg per kilogram body weight of the urea active therapeutic compound or compositions thereof are known to be useful in the treatment of the diseases, disorders, and conditions contemplated in the present inventive subject matter. Typically, this effective amount of the urea active agent will generally comprise from about 0.1 mg to about 100 mg per kilogram of patient body weight per day. Moreover, it will be understood that this dosage of active therapeutic agents can be administered in a single or multiple dosage units to provide the desired therapeutic effect.

If desired, other therapeutic agents can be employed in conjunction with those provided by the present inventive subject matter. The amount of active ingredients that may be combined with the carrier materials to produce a single dosage form will vary depending upon the host treated, the nature of the disease, disorder, or condition, and the nature of the active ingredients.

The present inventive compositions may be given in a single or multiple doses daily. In a preferred embodiment, the present inventive compositions are given from one to three times daily. Starting with a low dose twice daily and slowly working up to higher doses if needed is a preferred strategy. The amount of active ingredients that may be combined with the carrier materials to produce a single dosage form will vary depending upon the host treated, the nature of the disease, disorder, or condition, and the nature of the active ingredients.

It is understood, however, that a specific dose level for any particular patient will depend upon a variety of factors well known in the art, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular drug or drug combination and the desired dosage. See, for example, “Remington's Pharmaceutical Sciences”, 18th ed. (1990, Mack Publishing Co., Easton, Pa. 18042) and “Harry's Cosmeticology”, 8th ed. (2000, Chemical Publishing Co., Inc., New York, N.Y. 10016), the entire disclosures of which are hereby incorporated by reference. Such formulations may influence the physical state, stability, rate of in vivo release, and rate of in vivo clearance of the therapeutic agents.

### EXAMPLES

The following examples are illustrative of the present inventive subject matter and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates the preparation of a preferred cream of the present inventive subject matter:

| | % W/W |
|---|---|
| Castor Oil | 8.00 |
| Glyceryl Stearate (&) PEG-100 Stearate | 5.00 |
| Laureth-4 | 1.00 |
| Magnesium Aluminum Silicate | 2.50 |
| Polyoxyl 40 Stearate | 3.00 |
| Purified Water | 40.5 |
| Urea | 40.0 |
| | 100.0% |

1. An aqueous phase is prepared by mixing the urea in purified water for 20 minutes at a temperature of 25-30° C. The magnesium aluminum silicate is then slowly added to this mixture and then mixed for 60 minutes. Temperature is maintained at 25-30° C. After mixing, the temperature of the aqueous phase is raised to 70±2° C.

2. In a separate container, an oil phase is prepared by heating to 70±2° C. until melted the castor oil, the glyceryl stearate (&) PEG-100 stearate, the laureth-4, and the polyoxyl 40 stearate. These ingredients are then mixed until the oil phase is uniform in appearance.

3. The oil phase is then added to the aqueous phase at a temperature of 70±2° C. while mixing for 20 minutes to form an emulsion. The emulsion is then cooled at a rate which lowers the temperature to 25±2° C. within 3-8 hours.

### Example 2

The following example illustrates the preparation of another cream of the present inventive subject matter:

| | % W/W |
|---|---|
| Olive Oil | 30.0 |
| Glyceryl Stearate (&) PEG-100 Stearate | 7.50 |
| Polyoxyl 40 Stearate | 2.00 |
| Castor Wax | 1.00 |
| Magnesium Aluminum Silicate | 3.00 |
| Butylated Hydroxytoluene | 0.05 |
| Urea | 20.0 |
| Laureth-4 | 1.00 |
| Water | 35.45 |
| | 100.0% |

1. An oil phase is prepared by combining the Olive oil, Glyceryl Stearate (and) PEG-100 Stearate, Polyoxyl-40 Stearate, Butylated Hydroxytoluene and Castor Wax and heating until a temperature of 60 to 70° C. is reached. The Urea is then added to and dispersed in this mixture. The Magnesium Aluminum Silicate is then added to and dispersed in this mixture.

2. In a separate container, an aqueous phase is prepared by combining the Water and Laureth-4. This mixture is then heated to a temperature of 60 to 70° C. The remaining Magnesium Aluminum Silicate is then added to and dispersed in this mixture.

3. While mixing, the aqueous phase is added to the oil phase. The two phases are then mixed until cool, at a temperature of 15 to 30° C., to form an emulsion.

### Example 3

The following example illustrates the preparation of a gel of the present inventive subject matter:

| | % W/W |
|---|---|
| Safflower Oil | 15.0 |
| Propylene Glycol | 9.0 |
| Oleth-10 | 20.0 |
| PEG-25 Hydrogentated Castor Oil | 15.0 |
| Sorbitol | 6.9 |
| Urea | 5.0 |
| Disodium EDTA | 0.1 |
| Perfume | q.s. |
| Water | 29.0 |
| | 100.0% |

1. An oil phase is prepared by combining the Safflower Oil, Oleth-10, PEG-25 Hydrogenated Castor Oil, Propylene Glycol, and Sorbitol and heating until a temperature of 75±2° C. is reached.

2. In a separate container, an aqueous phase is prepared by combining the water and Disodium EDTA and heating to a temperature of 65 to 75° C. The urea is then added to and dispersed in this mixture.

3. The aqueous phase and the oil phase are combined and mixed until uniform. The emulsion is then cooled to a temperature of 20 to 30° C. The Perfume may be added as desired during cooling.

### Example 4

The following example illustrates the preparation of a skin cleanser of the present inventive subject matter:

| | % W/W |
|---|---|
| Isopropyl Palmitate | 3.7 |
| Oat Oil | 5.0 |
| Urea | 7.0 |
| Acetylated Lanolin Alcohol | 0.3 |
| Petrolatum | 1.0 |
| Nonoxynol-4 | 2.3 |
| Sodium Methyl Cocoyl Taurate | 23.0 |
| Sodium Nonoxynol-4 Sulfate | 6.4 |
| Sodium Chloride | 3.0 |
| Trisodium Phosphate | 1.0 |
| Water | 47.3 |
| | 100.0 |

1. An oil phase is prepared by combining the Isopropyl Myristate, Oat Oil, Acetylated Lanolin Alcohol, Petrolatum, and Nonoxynol-4 and heating until a temperature of 60 to 80° C. is reached. The Urea is then added to and dispersed in this mixture.

2. In a separately container, an aqueous phase is prepared by combining the Water, Sodium Methyl Cocoyl Taurate, Sodium Nonoxynol-4 Sulfate, Trisodium Phosphate, and Sodium Chloride and heating to a temperature of 60 to 80° C.

3. The aqueous phase and the oil phase are combining with mixing and then cooled to a temperature of 15 to 30° C.

### Example 5

The following example illustrates the preparation of a solution of the present inventive subject matter:

| | % W/W |
|---|---|
| Urea | 10.0 |
| Water | 27.0 |
| Canola Oil | 3.0 |
| Sorbitan Sequioleate | 9.0 |
| Ethanol | 49.5 |
| Polysorbate 80 | 1.0 |
| Dimethicone Copolyol | 0.5 |
| | 100.0 |

1. An aqueous phase is prepared by combining the Water and Urea. The ethanol, Polysorbate 80, and dimethicone copolyol are then added to this mixture while mixing.

2. In a separate contained, an oil phase is prepared by combining the Canola oil with the Sorbitan Sequioleate.

3. The oil phase is then added to the water mixture to form a solution.

### Example 6

| | % W/W |
|---|---|
| Water | 29.50 |
| Magnesium Aluminum Silicate | 2.50 |
| Xanthan gum | 0.50 |
| Urea | 50.00 |
| Castor Oil | 8.00 |
| Cetyl alcohol | 0.50 |
| Glyceryl Stearate (&) PEG-100 Stearate | 5.00 |
| Polyoxyl 40 Stearate | 3.00 |
| Laureth-4 | 1.00 |
| | 100.0% |

1. An aqueous phase is prepared by combining the Water and Urea. The magnesium aluminum silicate is added to this mixture while mixing. The xanthan gum further added while mixing continues. This mixture is then heated to a temperature of 60 to 75° C.

2. In a separate contained, an oil phase is prepared by combining the castor oil with the cetyl alcohol, the glyceryl stearate and PEG-100 stearate, the laureth-4, and the polyoxyl-40 stearate. This mixture is then heated to a temperature of 60 to 75° C.

3. The oil phase is then added to the water mixture and mixed at a temperature of 60 to 75° C. The mixture is then cooled while mixing to a temperature of 15 to 30° C.

### Example 7

A patient is suffering from dry skin. A pharmaceutical composition of the present inventive subject matter is topically administered to the patient. It would be expected that the patient would improve his/her condition or recover.

### Example 8

A patient is suffering from rough skin. A pharmaceutical composition of the present inventive subject matter is topically administered to the patient. It would be expected that the patient would improve his/her condition or recover.

### Example 9

A patient is suffering from dermatitis. A pharmaceutical composition of the present inventive subject matter is topically administered to the patient. It would be expected that the patient would improve his/her condition or recover.

### Example 10

A patient is suffering from psoriasis. A pharmaceutical composition of the present inventive subject matter is topically administered to the patient. It would be expected that the patient would improve his/her condition or recover.

### Example 11

A patient is suffering from xerosis. A pharmaceutical composition of the present inventive subject matter is topically administered to the patient. It would be expected that the patient would improve his/her condition or recover.

### Example 12

A patient is suffering from ichthyosis. A pharmaceutical composition of the present inventive subject matter is topically administered to the patient. It would be expected that the patient would improve his/her condition or recover.

### Example 13

A patient is suffering from eczema. A pharmaceutical composition of the present inventive subject matter is topically administered to the patient. It would be expected that the patient would improve his/her condition or recover.

### Example 14

A patient is suffering from keratosis. A pharmaceutical composition of the present inventive subject matter is topically administered to the patient. It would be expected that the patient would improve his/her condition or recover.

### Example 15

A patient is suffering from keratoderma. A pharmaceutical composition of the present inventive subject matter is topically administered to the patient. It would be expected that the patient would improve his/her condition or recover.

### Example 16

A patient is suffering from corns. A pharmaceutical composition of the present inventive subject matter is topically administered to the patient. It would be expected that the patient would improve his/her condition or recover.

### Example 17

A patient is suffering from calluses. A pharmaceutical composition of the present inventive subject matter is topically administered to the patient. It would be expected that the patient would improve his/her condition or recover.

The inventive subject matter being thus described, it will be apparent that the same may be modified or varied in many ways. Such modifications and variations are not to be regarded as a departure from the spirit and scope of the inventive subject matter, and all such modifications and variations are intended to be included within the scope of the following claims.

We claim:

1. A dermatological composition suitable for topical administration comprising:
about 5 to about 60% by weight of an active ingredient consisting of urea;
about 3 to about 45% by weight of a vegetable oil or a derivative thereof;
water; and
remaining amounts of one or more dermatologically acceptable agents.

2. The dermatological composition of claim 1, comprising about 30 to about 60% by weight of said active ingredient and about 3 to about 20% by weight of said vegetable oil or derivative thereof.

3. The dermatological composition of claim 1, comprising about 40 to about 55% by weight of said active ingredient and about 5 to about 15% by weight of said vegetable oil or derivative thereof.

4. The dermatological composition of claim 1, wherein said composition has a pH of about 3 to about 11.

5. The dermatological composition of claim 4, wherein said composition has a pH of about 5 to about 10.5.

6. The dermatological composition of claim 5, wherein said composition has a pH of about 7 to about 10.

7. The dermatological composition of claim 1, wherein said composition is an emulsion having an oil phase and an aqueous phase.

8. The dermatological composition of claim 7, formulated as an oil-in-water emulsion.

9. The dermatological composition of claim 7, wherein said vegetable oil or derivative thereof is present in said oil phase and is selected from the group consisting of castor oil, sunflower oil, a liquid fraction of karite butter, cottonseed oil, canola oil, corn oil, hydrogenated vegetable oil, peanut oil, sesame oil, soybean oil, palm oil, coconut oil, rapeseed oil, safflower oil, cocoa butter, linseed oil, olive oil, almond oil, avocado oil, citrus seed oil, cohune oil, tallow, oat oil, palm kernel oil, rice bran oil, tucum oil, babassu oil, derivatives thereof, and mixtures thereof.

10. The dermatological composition of claim 7, wherein said dermatologically acceptable agents are selected from the group consisting of emulsifiers, surfactants, suspending agents, antioxidants, chelates, emollients, humectants, fluid alkyl alcohols, thickening agents, polysiloxanes, modified polysiloxanes, pH modifiers, and mixtures thereof.

11. The dermatological composition of claim 10, wherein said emulsion is formed using an emulsifier selected from the group consisting of straight or branched chain fatty acids, polyoxyethylene sorbitan fatty acid esters, sorbitan fatty acid esters, propylene glycol stearate, glyceryl stearate, polyethylene glycol, fatty alcohols, polymeric ethylene oxide-propylene oxide block polymers, derivatives thereof, pharmaceutically acceptable salts thereof, and mixtures thereof.

12. The dermatological composition of claim 11, wherein said emulsifier is present in a phase of said emulsion selected from the group consisting of said oil phase, said aqueous phase, and a combination thereof.

13. The dermatological composition of claim 10, wherein said surfactant is selected from the group consisting of a nonionic surfactant, an anionic surfactant, an amphoteric surfactant, a cationic surfactant, and mixtures thereof.

14. The dermatological composition of claim 10, comprising at least two surfactants.

15. The dermatological composition of claim 10, wherein said suspending agent and said surfactant are present in a phase of said emulsion selected from the group consisting of said oil phase, said aqueous phase, and a combination thereof.

16. The dermatological composition of claim 1, comprising about 5 to about 90% by weight of water.

17. The dermatological composition of claim 7, wherein said urea is present in said aqueous phase or said oil phase.

18. The dermatological composition of claim 1, wherein said composition is in a lotion, cream, ointment, shampoo, gel, aerosol, solution, paste, suspension, skin cleanser, bar, or other pharmaceutically acceptable topical dosage form.

19. A method of treating a skin disorder in a mammal, comprising topically administering to skin of a mammal in need thereof a therapeutically effective amount of the dermatological composition of claim 1.

20. The method of claim 19, wherein said skin disorder is selected from the group consisting of cracked skin, disturbed barrier function, dry skin, eczema, icthyotic atopic eczema, acthyotic skin, subacute and chronic atopic eczema, xerosis, rough skin, dermatitis, psoriasis, ichthyosis, keratosis, keratoderma, corns, calluses, scales, nummular dermatitis, lichen simplex chronicus, pityriasis rosea, dandruff, acne, pruritus, age spots, lentigines, melasmas, wrinkles, warts, blemished skin, hyperpigmented skin, hyperkeratotic skin, inflammatory dermatoses, skin changes associated with aging, skin requiring cleansers, and combinations thereof.

21. The method of claim 19, wherein said topical administration moisturizes said skin.

22. The method of claim 19, wherein said method improves moisture content of said skin.

23. A method of treating or preventing dry skin in a mammal, comprising topically administering to skin of a mammal in need thereof a therapeutically effective amount of the dermatological composition of claim 1.

24. The method of claim 23, wherein said topical administration moisturizes said skin.

25. A dermatological composition suitable for topical administration comprising an oil-in-water emulsion comprising:
an oil phase comprising about 3 to about 20% by weight of the overall weight of the composition of a vegetable oil or a derivative thereof and at least one emulsifier; and
an aqueous phase comprising about 30 to about 60% by weight of the overall weight of the composition of an active ingredient consisting of urea and about 15 to about 55% by weight of the overall weight of the composition of water,
wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

26. The dermatological composition of claim 25, comprising about 45 to about 55% by weight of said active ingredient and about 5 to about 10 by weight of said vegetable oil or derivative thereof.

27. The dermatological composition of claim 25, wherein said composition has a pH of about 7 to about 10.

28. A method of treating a skin disorder in a mammal, comprising topically administering to skin of a mammal in need thereof a therapeutically effective amount of the dermatological composition of claim 25.

29. A method of treating or preventing dry skin in a mammal, comprising topically administering to skin of a mammal in need thereof a therapeutically effective amount of the dermatological composition of claim 25.

30. A dermatological composition suitable for topical administration comprising an oil-in-water emulsion comprising:
an oil phase comprising about 15 to about 45% by weight of the overall weight of the composition of a vegetable oil or a derivative thereof and at least one emulsifier; and
an aqueous phase comprising about 40 to about 55% by weight of the overall weight of the composition of an active ingredient consisting of urea and about 25 to about 40% by weight of the overall weight of the composition of water,
wherein said emulsion further comprises at least two surfactants and at least one suspending agent present in a phase of said emulsion selected from the group consisting of said oil phase, said aqueous phase, and a combination thereof, and wherein said dermatological composition has a pH of from about 3 to about 11.

31. The dermatological composition of claim 30, comprising about 47 to about 53% by weight of said active ingredient and about 5 to about 10% by weight of said vegetable oil or derivative thereof.

32. A method of treating a skin disorder in a mammal, comprising topically administering to skin of a mammal in need thereof a therapeutically effective amount of the dermatological composition of claim 30.

33. A method of treating or preventing dry skin in a mammal, comprising topically administering to skin of a mammal in need thereof a therapeutically effective amount of the dermatological composition of claim 30.
